(19) United States
(12) Patent Application Publication
(10) Pub. No.: US 2022/0082277 A1
(43) Pub. Date: Mar. 17, 2022

(54) Air Vent Remora Air Filtering System

(71) Applicant: Angelica Lowe, Westworth Village, TX (US)

(72) Inventor: Angelica Lowe, Westworth Village, TX (US)

(21) Appl. No.: 17/019,198

(22) Filed: Sep. 12, 2020

(51) Int. Cl. F24F 8/50; F24F 13/28; B01D 46/00; A61L 9/014; A61L 9/015

(52) U.S. Cl. CPC F24F 8/50; F24F 13/28; B01D 46/0006; B01D 46/0038; A61L 9/014; A61L 2209/15; B01D 2275/205; B01D 2279/50; A61L 2209/14; A61L 2209/16; A61L 2209/22; A61L 9/015

(57) ABSTRACT

An indoor external air filtering and fragrance diffusing system composed of a cage like apparatus that attaches to an existing air vent and holds in place air filter. The present invention includes a device to hold a filter from the exterior of a conventional air vent, allowing the airflow into a room from the central air system and a customized air filter. The present invention incorporates a variety of air filters that can be adapted to the specific needs of a user. One variation of the filter contains an embodiment that is comprised of absorbent material where a fragrance of choice can be added. Yet another variation of the filter contains an embodiment of absorbent material that already contains fragrance.

### BACKGROUND OF THE INVENTION

### Field of Invention

The present invention is in field of indoor air filtration, and indoor air freshening including air filters and fragrance diffuser. It pertains to methods and apparatus to hold a filter from the exterior of a conventional air vent, allowing the airflow into a room from the central air system.

### Description of Related Art

HVAC stands for heating, ventilation and air conditioning system, it provides heating and cooling to residential and commercial buildings. These systems use fresh air from outdoors to provide indoor air quality throughout the process of ventilation which replaces or exchanges the air within a particular space, it involves the removal of moisture, smoke, odors, heat dust, airborne bacteria, carbon dioxide, and other gases such radon, it also provides control of temperature and oxygen.

In general, the HVAC system is comprised of nine elements: Air return, Filter, Exhausts outlets, Ducts, Electrical elements, Outdoor unit, Compressor, Coils and Blower. The Air Return is the part of the HVAC system that marks the starting point of the ventilation cycle. This return sucks in air, draws it through a filter, and then passes it into the main system.

The filter is the second part of the air return in which the air is drawn through.

The exhaust outlets are where the exhaust created by the heating system is expelled.

Ducts are the channels in which the heated or cooled air passes through.

Electrical components such a thermostat or a braker.

The Outdoor Unit houses the fan which provides air flow.

The Compressor is responsible for converting refrigerant from a gas to liquid and sends it to the coils.

Coils cool the air as it passes through with a little help from the refrigerant.

The Blower draws in warm air through the main section of the unit.

Nevertheless, after HVAC system is installed and working properly, the reality shows that the indoor air quality is directly dependable on the Air Filter.

Great variety of air filters already exist that help to capture varying sizes of particles such as: lint, pollen, dust, pet dander, dead skin cells, smoke, smog, cough and sneeze particles, bacteria, virus, candle soot and air pollution in general.

The three air filter rating systems differ in ranges and were created by separate organizations.

MERV ratings extend from 1 to 16.

MPR measures ratings based on sizes 0.3 to 1 microns.

FPR ratings are on a scale from 4 through 10.

The MERV filter rating is the domestic and international industry standard rating system established by the American Society of Heating, Refrigerating, and Air Conditioning Engineers. The other two systems were created by “3M” (MPR) and “The Home Depot” (FPR) to differentiate their offerings.

However, a challenge with before mentioned system is that the indoor air quality depends on the efficiency of the Air Filter which depends on the quality and specifications of the filter itself, but its effectiveness is dependent on the frequency in which this filter is replaced.

Another challenge is that traditional HVAC filters are problematic to access and difficult to replace. The fact that they are hard to view makes it easy to forget about them and the well-timed and adequate replacement becomes compromised.

Another challenge is that the indoor air quality oft the whole house depends on a single air filter since most HVAC systems only required a single filter per unit that typically is located at the Air Return.

The HVAC system can be working properly but if the filter is saturated with excess of particles and contaminants, sooner or later these particles are returned into the ducts and reexpelled into the living areas and a cycle of indoor air contamination sets in motion. Thus, a greater problem is created and characterized as significant risk to the public from indoor environmental contaminants that are commonly found in homes, schools, offices and commercial buildings.

Most Americans spend 90 percent of the time indoors, even more so during COVID-19 Pandemic.

The United States Environmental Protection Agency has identified that the levels of indoor air pollution are often two to five times higher, and occasionally 100 times higher that outdoor levels. It estimates that the economic cost associated with adverse health and productivity effects of poor indoor air quality fall between $13 and $32 billion annually. Lung cancer, Asthma, and Allergies are the most common diseases associated with indoor air pollution.

Additionally, clogged air filters increase energy consumption by 5 to 15 percent. Therefore, it is evident that what is promptly needed is an external air filtering system that easily attaches to the existing air vent that solves the above limitations.

In some cases, a filter material in the form of roll is known which can be glue or attached to the air vent still remains impractical because it has to be cut to fit vent and can be easily detach causing problems to the user, therefore it does not solve the aforementioned problems.

Furthermore, the purpose of the present invention in addition to filter the air before it enters a room, it is also to impregnate with a fragrance or essential oils if the user decides to do so.

US Patent No.: 2010/0227545A1 Discloses an air vent cover and filter that is affixed to a frame that is attached to the flat portion of the air vent. The purpose of this invention is to filter the air before it enters a room, it also can be impregnated with a fragrance however, based on information available of such invention, it does not solve the problem of hassle-free operation and is not aesthetically inconspicuous. Additionally, it does not offer the capability of using different types of filters for customization of indoor air quality depending on the needs of a particular living area according to homeowner's desire.

### BRIEF SUMMARY OF THE INVENTION

The problems previously stated make difficult for houses, schools, offices and commercial buildings to enjoy of clean and unpolluted indoor air.

A strong need exists from homeowners, schools, offices and commercial buildings with a central air system, to have the option for an external air filter system that can aid to prevent dirt and dust particles that are circulating throughout the home and have infiltrated to the ductwork of the house, from reentering into the living spaces, therefore polluting the air that their dwellers breath.

In like manner a strong need exists from homeowners, schools, offices and commercial buildings with a central air system, to have the option for an external air filter system to to have the option to customize the intensity of the air filtration on each individual living area where an air vent exists by choosing specific air filter capabilities according to the desired level of filtration.

In like manner a strong need exists from homeowners, schools, offices and commercial buildings with a central air system, to have the option for a fragrance of choice carrier system that can aid in delivering such fragrance into a room or living space of choice. Even more so, the present COVID-19 Pandemic urges for a system that can help to improve the indoor air quality by minimizing the pollutants that enter a living space. The present inventor realized in an inventive moment that if, an air filter could be placed on each air vent, air filtration could be optimized by adding air filtration sites throughout the home instead of exclusively relying on a single air filter for the whole house. The inventor therefore constructed a rectangular frame fixture, with an opening where air filter can be inserted, the inventor realized that the present invention was not a replacement of the regular air filter link to the HVAC System, furthermore the invention, was an excellent complement to the existing HVAC System. The inventor concluded that both air filtering systems one internal and the other external could work in a synergistic way enhancing the quality of the indoor air, The inventor realized in an inventive moment that the relationship existing between both air filtration systems was observed in nature, most commonly between a shark and a remora fish, therefore the inventor determined that the big central unit filter could be referred as a shark and the small external filter could be referred as a remora fish, therefore the inventor decided to name his invention Air Vent Remora, however the present invention can also be referred simply as a filter holder.

Accordingly, strong magnets were embedded on each side of the back of the frame fixture to attach it to the flat surface of an existing air vent, although other methods for attaching the air vent fixture can be used such as suction cups, adhesive materials or screws.

According to one aspect of the present invention, the face or front of the filter holder, is designed to look like a regular air vent to appear inconspicuous to the eye and non-alter the aesthetics of the living area where it is placed.

Another embodiment of the present inventions is comprised of a custom fitted air filter that easily is inserted into the filter holder.

Yet another embodiment of the present inventions is comprised of a custom fit filter like absorbent fabric material where the fragrance of choice or even essential oils can be dispersed throughout the absorbent material so it can be diffused into the air of the living space of choice.

The present invention is shown in a rectangular shape, since most common air vents are rectangular in shape, however it can be conformed to a circular or a square shape as well, to fit the different air vent shapes.

### BRIEF DESCRIPTION OF THE DRAWINGS

FIG. 1A shows a filter holder also known as Air Vent Remora.

FIG. 1B shows a custom-made filter to slide into the filter holder of FIG. 1A.

FIG. 2. Shows another custom-built air filter, comprising an air filter medium 202, surrounded by a cardboard frame 201.

FIG. 3 Shows a custom-built air filter, comprising an air filter medium 302, and absorbent material 303 surrounded by a cardboard frame 301.

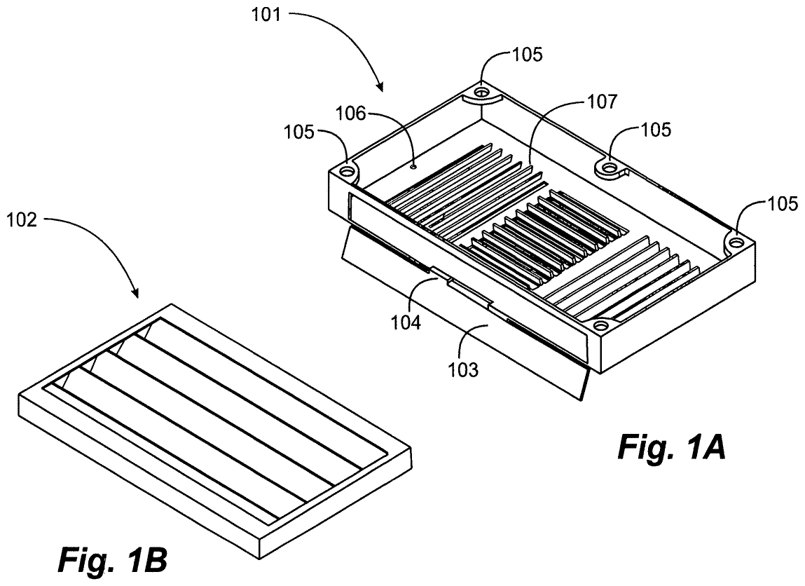

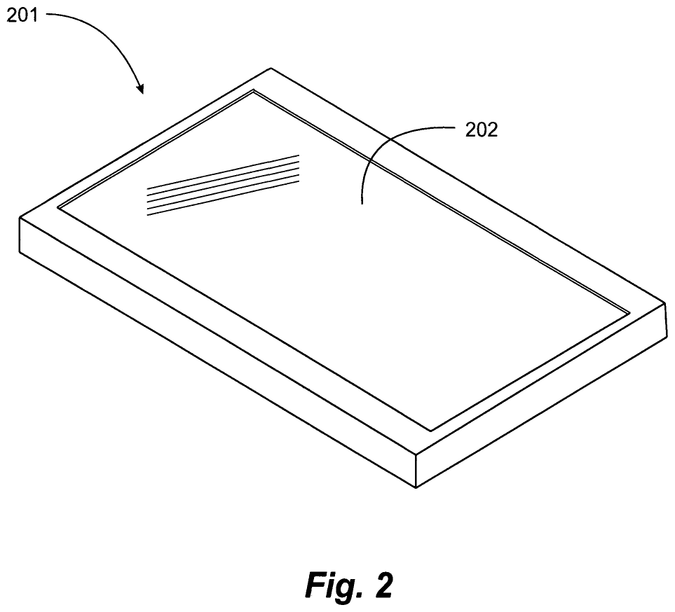

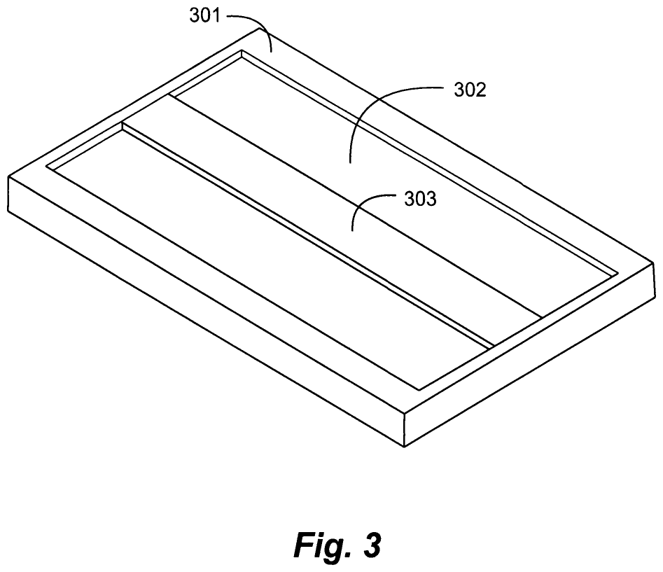

### DETAILED DESCRIPTION OF THE INVENTION

The present invention provides an external indoor air filter system attached to the conventional air vent that allows the airflow into a room from the central air system. The invention also enables users to enjoy the therapeutic benefits of essential oils or add the fragrance of choice into a room or designated living space. The invention consists of three (3) main components:
A frame fixture that is attached to the flat portion of a regular air vent.
An attachment medium, which may be magnets, suction cups, adhesive or screws.
A filter that is placed inside the frame fixture.
A hybrid filter with a combination of air filtering material and absorbent material.

The frame fixture, hereafter referred as Air Vent Remora, is aesthetically similar to the actual air vent, 101, that is to say, a “faux air vent” that fits over the flat portion of the regular air vent, it has a horizontal sliding compartment and a spring-loaded door 103 that holds the air filter in place for as long as it is needed or desired and makes it simple and straightforward to change the filter.

The frame fixture's main function is to hold the filter in place. It can be attached to the conventional air vent in various ways, such as with magnets 105, suction cups, but it also can be done with the use of an adhesive material or with the aid of a screws 106. once the frame is attached to the air vent, it can stay in place indefinitely. It can be conformed to any size and shape that best suits the existing air vent.

The air filter can be made of a spun fiberglass material or from pleated paper or cloth enclosed in a cardboard frame 102.

The filter can also be made of nonwoven fabric with absorbent qualities to hold fragrance or essential oils that is glued to a plastic border 301 to hold its shape and adds rigidity so it can be easily inserted in the horizontal sliding slot otherwise stated as sliding compartment of the frame fixture.

The filter can be reinforced in certain spots to make it highly absorbent so an essential oil or fragrance of choice can be added to the filter and carried into the living space through the air flow.

The filter can be pre-scented with a wide variety of fragrances to choose from or can be unscented to allow higher level of customization of the air that circulates into the living space.

The Air Vent Remora as an external indoor air filtering system improves the indoor air quality of an entire house, school, office or commercial building with a Central Air Unit or (HVAC System) by adding an extra layer of air filtration as it decreases the number of particles of pollutants circulating in the air.

The Air Vent Remora as an external indoor air filtering system facilitates air filtering customization by providing different levels of air filtration throughout a house, school, office or commercial building with a Central Air Unit or (HVAC System by granting the opportunity to use the MERV filter ratings of 6, 8, 11, or 13 levels depending on the living area in question. For example, an individual may choose to use a filter MERV level 6 in a less transited living space, MERV level 8 in the laundry room, MERV level 11 in the kitchen and MERV level 13 in the bedrooms.

The Air Vent Remora is an external indoor air freshener system that improves the indoor air quality of an entire house, school, office or commercial building with a Central Air Unit or (HVAC System) by customizing the ambience of a specific living area where an air vent is located, by allowing fragrance to be infuse into the air.

The Air Vent Remora is an external indoor air freshener system that carries essential oil or fragrance and improves the indoor air quality of an entire house, school, office or commercial building with a Central Air Unit or (HVAC System) by customizing the ambience of a specific living area where an air vent is located, by allowing essential oils or fragrance to be infuse into the air.

The Air Vent Remora is an external indoor air filtering and freshener system that improves the indoor air quality of an entire house, school, office or commercial building with a Central Air Unit or (HVAC System) in a hassle-free manner due to its simplicity. The Air Vent Remora is an external indoor air filtering and freshener system that improves the indoor air quality of an entire house, school, office or commercial building with a Central Air Unit or (HVAC System) in an aesthetically pleasing manner due to its inconspicuous design.

The Air Vent Remora is an external indoor air filtering and freshener system that improves the indoor air quality of an entire house, school, office or commercial building with a Central Air Unit or (HVAC System) by promoting air filter timely replacement due to its external nature and easy accessibility.

The claims are listed below with status indication:

1. An indoor air filtering and aroma diffusing system comprising:
a rectangular frame fixture providing a filter holder with an attachment interface to an existing air vent; and
an air filter sized to be inserted into the rectangular frame fixture through a spring-loaded door located on one side of the rectangular frame fixture.

2. The indoor air filtering and aroma diffusing system of claim 1 wherein the attachment interface comprises a plurality of magnets in or on one side of the rectangular frame, positioned to hold the rectangular frame against a flat portion of an existing air vent.

3. The indoor air filtering and aroma diffusing system of claim 1 wherein the attachment interface comprises a plurality of suction cups in or on one side of the rectangular frame, positioned to hold the rectangular frame against a flat portion of an existing air vent.

4. The indoor air filtering and aroma diffusing system of claim 1 wherein the attachment interface comprises an adhesive material on a surface of the rectangular frame, adhering the rectangular frame to a flat portion of an existing air vent.

5. The indoor air filtering and aroma diffusing system of claim 1 wherein the attachment interface comprises conventional screw fasteners through a surface of the rectangular frame, fastening the rectangular frame to a flat portion of an existing air vent.

6. The indoor air filtering and aroma diffusing system of claim 1 wherein the air filter is formed from spun fiberglass material.

7. The indoor air filtering and aroma diffusing system of claim 1 wherein the air filter is formed from pleated paper or cloth enclosed in a cardboard frame.

8. The indoor air filtering and aroma diffusing system of claim 1 wherein the air filter comprises a portion of a fabric absorbing fragrance material or essential oils.

9. The indoor air filtering and aroma diffusing system of claim 1 wherein the air filter has one or more indented regions for insertion of absorbent material holding fragrance material or essential oils.

10. A method for adding filtration to an existing air vent, comprising:
forming a rectangular frame fixture providing a filter holder with an attachment interface to the existing air vent;
joining the rectangular frame fixture to a flat portion of the existing air vent; and
inserting an air filter into the rectangular frame fixture through a spring-loaded door located on one side of the rectangular frame fixture.

11. The method of claim 10 wherein the attachment interface comprises a plurality of magnets in or on one side of the rectangular frame, positioned to hold the rectangular frame against a flat portion of an existing air vent, further comprising placing the side of the rectangular frame against the flat portion of the air vent such that the rectangular frame is held by the magnets to the air vent.

12. The method of claim 10 wherein the attachment interface comprises a plurality of suction cups in or on one side of the rectangular frame, positioned to hold the rectangular frame against a flat portion of an existing air vent, further comprising placing the side of the rectangular frame having suction cups against the flat portion of the air vent such that the rectangular frame is held by the suction cups to the air vent.

13. The method of claim 10 wherein the attachment interface comprises an adhesive material on a surface of the rectangular frame, adhering the rectangular frame to a flat portion of an existing air vent, further comprising placing the side of the rectangular frame having adhesive material against the flat portion of the air vent such that the rectangular frame is held by the adhesive material to the air vent.

14. The method of claim 10 wherein the attachment interface comprises conventional screw fasteners, further comprising fastening the rectangular frame to a flat portion of an existing air vent with the screw fasteners.

15. The method of claim 10 further comprising forming the air filter from spun fiberglass material.

16. The method of claim 10 comprising forming the air filter from pleated paper or cloth enclosed in a cardboard frame.

17. The method of claim 10 comprising forming the air filter with a portion of a fabric absorbing fragrance material or essential oils.

18. The method of claim 10 comprising forming the air filter with one or more indented regions for insertion of absorbent material holding fragrance material or essential oils.
